(19) United States
(12) Patent Application Publication
(10) Pub. No.: US 2025/0339131 A1
(43) Pub. Date: Nov. 6, 2025

(54) METHODS AND APPARATUS TO MEASURE TRANSISTOR TEMPERATURE

(71) Applicant: Texas Instruments Incorporated, Dallas, TX (US)

(72) Inventors: Savyan Kanisserry, Bangalore (IN); Ravikumar Pattipaka, Bangalore (IN); Jeevan Mithra, Bangalore (IN); Vajeed Nimran, Bangalore (IN); Shabbir Amjherawala, Bangalore (IN)

(21) Appl. No.: 18/932,290

(22) Filed: Oct. 30, 2024

(30) Foreign Application Priority Data
May 6, 2024 (IN) 202441035738

(51) Int. Cl. A61B 8/00; A61B 8/08; G01K 3/00; G01K 7/00

(52) U.S. Cl. CPC A61B 8/546; A61B 8/485; G01K 3/005; G01K 7/00

(57) ABSTRACT

Example methods and apparatus to measure transistor temperature are described herein. An example apparatus includes a silicon signal layer and a metal layer over the silicon signal layer, wherein the metal layer includes a first metal region that is electrically coupled to the silicon signal layer and a second metal region that is electrically decoupled from the silicon signal layer.

### RELATED APPLICATION

This patent claims the benefit of Indian Provisional Application No. 20/244,1035738, which was filed on May 6, 2024. Indian Provisional Application No. 20/244,1035738 is hereby incorporated herein by reference in its entirety. Priority to Indian Provisional Application No. 20/244,1035738 is hereby claimed.

### TECHNICAL FIELD

This description relates generally to temperature measurement and, more particularly, to methods and apparatus to measure transistor temperature.

### BACKGROUND

Transmitters are electrical devices that transmit, project, or output analog signals. Transmitters are used in a wide range of fields including medical imaging, telecommunications, data transfer, and other fields that utilize analog signals. For example, medical imaging devices, e.g., ultrasound devices, utilize a transmitter to transmit an ultrasonic analog signal to an object, e.g., an organ. The medical imaging device obtains from the object a reflection of the transmitted analog signal after transmitting the output analog signal and processes the obtained reflected signal to generate an image of the object.

### SUMMARY

A first example integrated circuit includes a silicon signal layer; and a metal layer over the silicon signal layer, wherein the metal layer includes a first metal region that is electrically coupled to the silicon signal layer and a second metal region that is electrically decoupled from the silicon signal layer.

A second example integrated circuit includes a silicon substrate; a buried oxide region on the silicon substrate; a silicon signal layer on the buried oxide region; a metal layer over the silicon signal layer, wherein the metal layer includes: a first metal region that is electrically coupled to the silicon signal layer; and a second metal region that is electrically decoupled from the silicon signal layer; and oxide material implemented: a) between the first metal region and second metal region and b) between the second metal region and the silicon signal layer.

An example ultrasound device includes: an integrated circuit including: a silicon substrate; a buried oxide region on the silicon substrate; a silicon signal layer on the buried oxide region, wherein the silicon signal layer includes a high voltage transistor; a metal layer over the silicon signal layer, wherein the metal layer includes a first metal region that is electrically coupled to the high voltage transistor and a second metal region that is electrically decoupled from the high voltage transistor; and oxide material implemented: a) between the first metal region and second metal region and b) between the second metal region and the silicon signal layer; detector circuitry coupled to the second metal region, the detector circuitry configured to detect when a temperature of the high voltage transistor exceeds a threshold temperature; and control circuitry coupled to the detector circuitry, the control circuitry configured to perform one or more heat reduction operations responsive to a detection from the detector circuitry.

### BRIEF DESCRIPTION OF THE DRAWINGS

FIG. 1 is a block diagram of an example environment of an ultrasound system.

FIG. 2 is a block diagram of an example implementation of the transmitter circuitry of FIG. 1.

FIG. 3A is a top-down view of an Integrated Circuit (IC) that implements the High Voltage (HV) transistor and detector circuitry of FIG. 2 using a previous approach.

FIG. 3B is a top-down view of an Integrated Circuit (IC) that implements the High Voltage (HV) transistor and detector circuitry of FIG. 2 using examples described herein.

FIG. 4 is a schematic diagram of an example implementation of the detector circuitry, sense resistor, and HV transistor of FIGS. 2 and 3B.

FIG. 5 is a cross-sectional view of a first region of the example IC of FIG. 3B.

FIG. 6 is an example representation of heat transfer between the HV transistor, metal layers, and sense resistor of FIG. 5.

FIG. 7 is a cross-sectional view of a second region of the example IC of FIG. 3B where a silicon bump is implemented over the sense resistor.

FIG. 8 is a flowchart representative of example machine-readable instructions or example operations that may be at least one of executed, instantiated, or performed the using the detector circuitry of FIG. 4 and the control circuitry of FIG. 1.

FIG. 9 is a flowchart representative of example machine-readable instructions or example operations that may be at least one of executed, instantiated, or performed the using the detector circuitry and the control circuitry of FIG. 1.

FIG. 10 is a block diagram of an example processing platform including programmable circuitry structured to execute, instantiate, or perform the example machine-readable instructions or perform the example operations of FIGS. 8 and 9 to implement the control circuitry 108 of FIG. 1.

The drawings are not necessarily to scale. Generally, the same reference numbers in the drawing(s) and this description refer to the same or similar (functionally and/or structurally) features and/or parts. Although the drawings show regions with clean lines and boundaries, some or all of these lines and boundaries may be idealized. In reality, the boundaries or lines may be unobservable, blended or irregular.

### DETAILED DESCRIPTION

Medical imaging devices, such as ultrasound devices, utilize transmitted and reflected analog signals to generate images of objects, e.g., organs. For example, an ultrasound device uses transmitter circuitry to transmit ultrasonic wave pulses to the object. After transmitting the ultrasonic waves, the ultrasound device uses a receiver to obtain a reflected echo wave and process the obtained echo wave to image the object.

Ultrasound devices can operate in a variety of different modes. For example, an elastography mode refers to a mode in which high power sound waves are continuously sent to the object, e.g., tissues inside a body. The tissues vibrate, responsive to the continuous signal, with an elasticity that can be indicative of whether the tissue is cancerous. The transmitter circuitry generates significant amounts of heat during the continuous generation of the high-power signal. In one example, an elastography mode transmission with a duration of 600microseconds (μs) can raise portions of such transmitter circuitry to 172 degrees Celsius (° C.). More generally, heat generated during elastography mode has the potential to damage one or more components of the ultrasound device if the signal is transmitted for an incorrect duration, at an incorrect power level, or another type of error occurs. Therefore, safety requirements for ultrasound systems use techniques to detect when the transmitter circuitry is about to overheat.

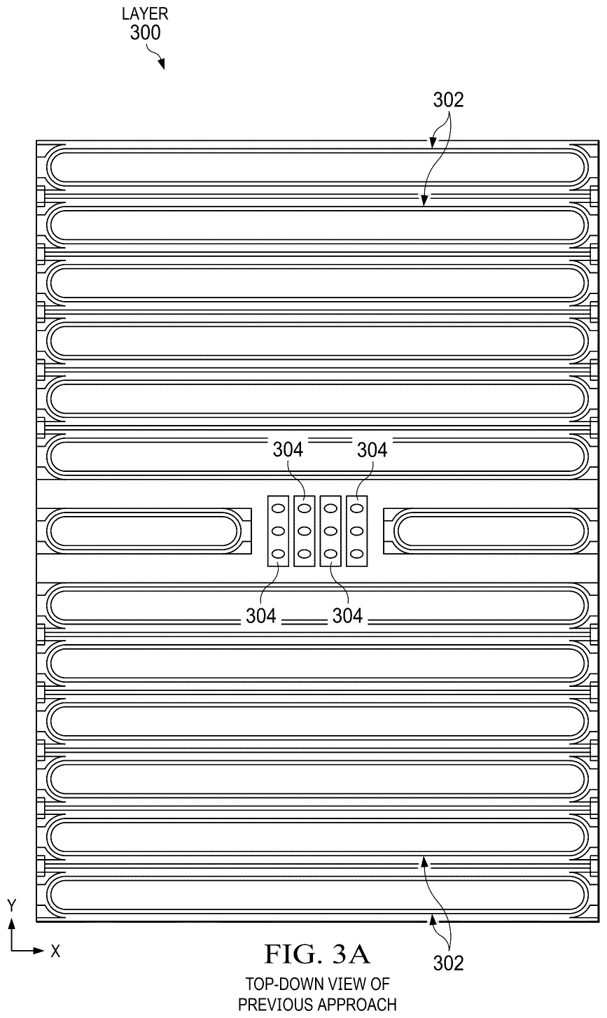

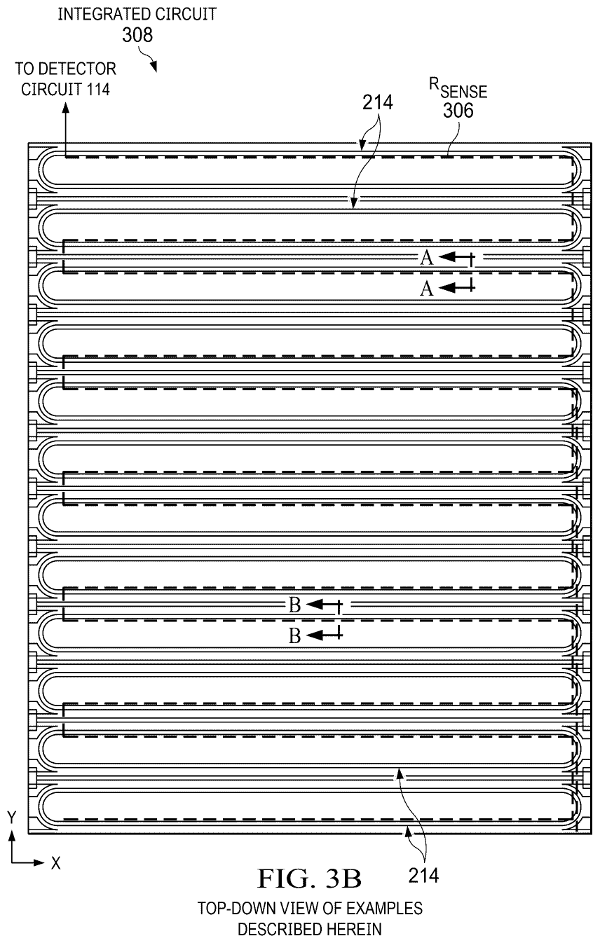

Previous approaches to detect the temperature of transmitter circuitry implement a bandgap core device that compares a first current source that changes proportionally to temperature with a second current source that changes inversely proportional to temperature. To ensure the current sources are responsive to temperature, the transmitter circuitry is implemented around the bandgap core device so that the bandgap core is implemented within the same three-dimensional portion of an Integrated Circuit (IC) that encapsulates the transmitter circuitry. The layout of such a previous approach is described further in connection with FIG. 3A.

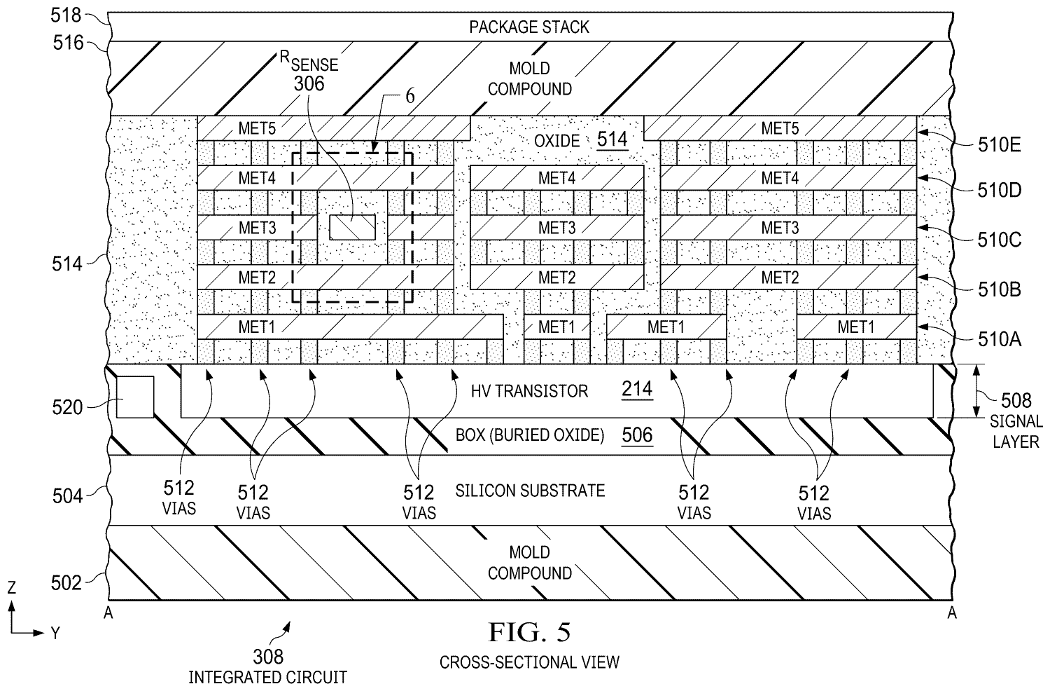

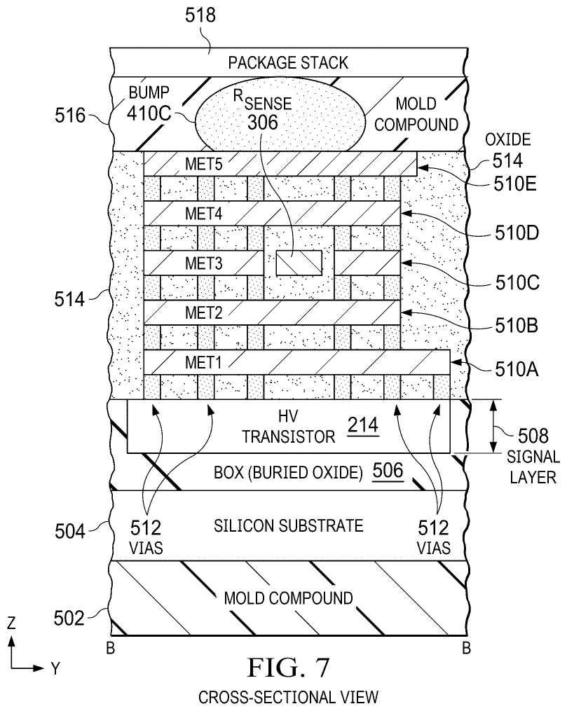

Recently, designers and manufacturers of ultrasound systems have begun to implement transmitter circuitry using a Silicon on Insulator (SOI). SOI refers to a semiconductor fabrication architecture that separates two layers of silicon with an insulator layer. The SOI architecture galvanically isolates one or more components of the transmitter circuitry from other components implemented within the IC, thereby protecting the other components from the high voltages used by the transmitter circuitry. The SOI architecture is described further in connection with FIGS. 5 and 7.

The insulation layer used in the SOI architecture is also a low thermal conductivity material that physically separates components from one another. As a result, a bandgap core device implemented in an IC with a SOI architecture cannot accurately detect the temperature of the surrounding component. For example, experiments have recorded a temperature difference of approximately 80° C. between a heat generating component and a bandgap core due to the isolation layer that is implemented in between them. Therefore, previous approaches to detect the temperature of ultrasound transmitter circuitry cannot support SOI architectures.

Example methods, apparatus, and systems described herein implement a technique to accurately detect the temperature of a heat generating component within an SOI architecture. An example IC includes one or more metal layers implemented over and electrically connected to a silicon signal layer. The example IC also includes a sense resistor (R_{SENSE}) that is surrounded by, but electrically disconnected from, the metal layers. The silicon signal layer implements a component, e.g., a component within ultrasound transmitter circuitry during elastography mode, which generates heat when operating. While a comparatively small amount of heat flows through the insulation layer that is implemented below and beside the silicon signal layer, a comparatively large amount of heat flows through the metal layers over the silicon signal layer. The heat then flows into the metal that implements R_{SENSE}, thereby changing the resistance value of R_{SENSE} proportionally to the temperature of the silicon signal layer.

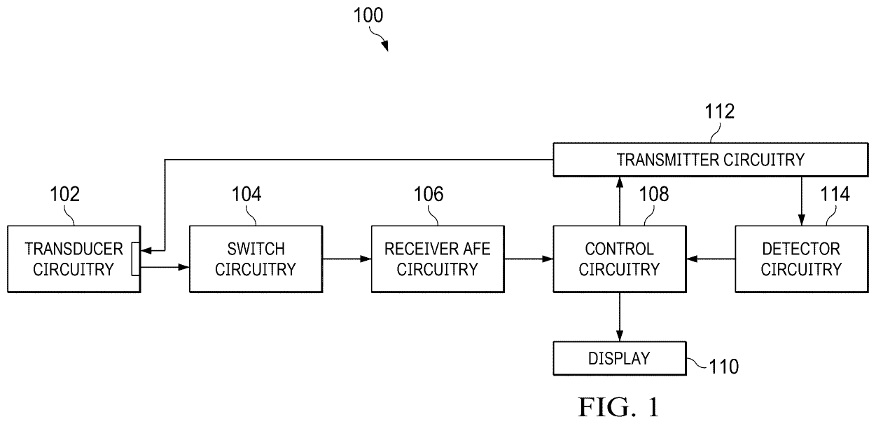

FIG. 1 is a block diagram of an example ultrasound system 100. In the example of FIG. 1, the ultrasound system 100 includes transducer circuitry 102, switch circuitry 104, receiver analog front-end (RX AFE) circuitry 106, control circuitry 108, a display 110, transmitter (TX) circuitry 112, and detector circuitry 114.

The control circuitry 108 has at least a first terminal, a second terminal, a third terminal, and a fourth terminal. The first terminal of the control circuitry 108 is coupled to the RX AFE circuitry 106. The second terminal of the control circuitry 108 is coupled to the display 110. The third terminal of the control circuitry 108 is coupled to the TX circuitry 112. The fourth terminal of the control circuitry 108 is coupled to detector circuitry 114. The control circuitry 108 may be instantiated, e.g., creating an instance of, bring into being for any length of time, materialize, implement, etc., by programmable circuitry such as a Central Processor Unit (CPU) executing first instructions. Also or alternatively, the control circuitry 108 may be instantiated, e.g., creating an instance of, bring into being for any length of time, materialize, implement, etc., by (i) an Application Specific Integrated Circuit (ASIC) or (ii) a Field Programmable Gate Array (FPGA) structured or configured in response to execution of second instructions to perform operations corresponding to the first instructions. Some or all of the circuitry of FIG. 1 may, thus, be instantiated at the same or different times. Some or all of the circuitry of FIG. 1 may be instantiated, for example, in one or more threads executing concurrently in hardware or in series in hardware. Moreover, in some examples, some or all of the circuitry of FIG. 1 may be implemented by microprocessor circuitry executing instructions or FPGA circuitry performing operations to implement one or more virtual machines or containers.

The control circuitry 108 manages the operations of the other components in the ultrasound system 100. For example, the control circuitry 108 switches the ultrasound system 100 between transmit mode and receive mode. In transit mode, the control circuitry 108 provides instructions to the TX circuitry 112, via the second terminal, that describe when and how to transmit a signal. The control circuitry 108 determines the instructions for the TX circuitry 112 responsive to inputs received from one or more of the RX AFE circuitry 106 and the detector circuitry 114. The control circuitry 108 may also interpret the input data from the RX AFE circuitry 106 and the detector circuitry 114, perform one or more signal processing operations, and instruct the display 110 to present results of the signal processing operations.

The transmitter circuitry 112 has a first terminal coupled to the control circuitry 108 and a second terminal coupled to the transducer circuitry 102. The TX circuitry 112 generates, responsive to instructions from the control circuitry 108, a high voltage signal that excites the transducer circuitry 102. In some examples, the TX circuitry 112 outputs a signal having a scale of approximately +/−100 Volts (V).

The transducer circuitry 102 has a terminal coupled to both the TX circuitry 102 and the switch circuitry 104. In transmit mode, the transducer circuitry 102 converts the high voltage signal produced by the TX circuitry 112 into ultrasound waves. The transducer circuitry 102 then emits the ultrasound waves into a target object, e.g., an organ. In receiving mode, the transducer circuitry 102 also converts incoming ultrasound waves, e.g., the wave that echoes off the target object, into a voltage signal. The transducer circuitry 102 may be implemented using any number of channels.

The switch circuitry 104 has a first terminal coupled to the transducer circuitry 102 and a second terminal coupled to the RX AFE circuitry 106. In transit mode, the switch circuitry 104 is turned OFF, e.g., acts as an open circuit, to protect the RX AFE circuitry 106 from the high voltage signal. In receiving mode, the switch circuitry 104 is turned ON, e.g., acts as a short circuit, to connect the transducer circuitry 102 to the RX AFE circuitry 106. Accordingly, the RX AFE circuitry 106 receives an echo from the transducer circuitry 102 during receiving mode.

The RX AFE circuitry 106 has a first terminal coupled to the switch circuitry 104 and a second terminal coupled to the control circuitry 108. The RX AFE circuitry 106 converts the analog echo signal obtained during receiving mode into digital data that is interpretable by the control circuitry 108.

The display 110 has a terminal coupled to the control circuitry 108. In some examples, the display 110 is illustrated or described as an external device. For example, when the control circuitry 108 is structured to use a wireless communication protocol to interface with the display 110, the display 110 may be an external device, such as a smartphone, tablet, screen, etc. In some examples, the control circuitry 108 is communicatively coupled to the display 110 using interface circuitry. In such examples, the interface circuitry implements one or more communication protocols to communicate with the display 110. For example, the control circuitry 108 uses a wireless communication protocol, e.g., Bluetooth, to communicate with the display 110.

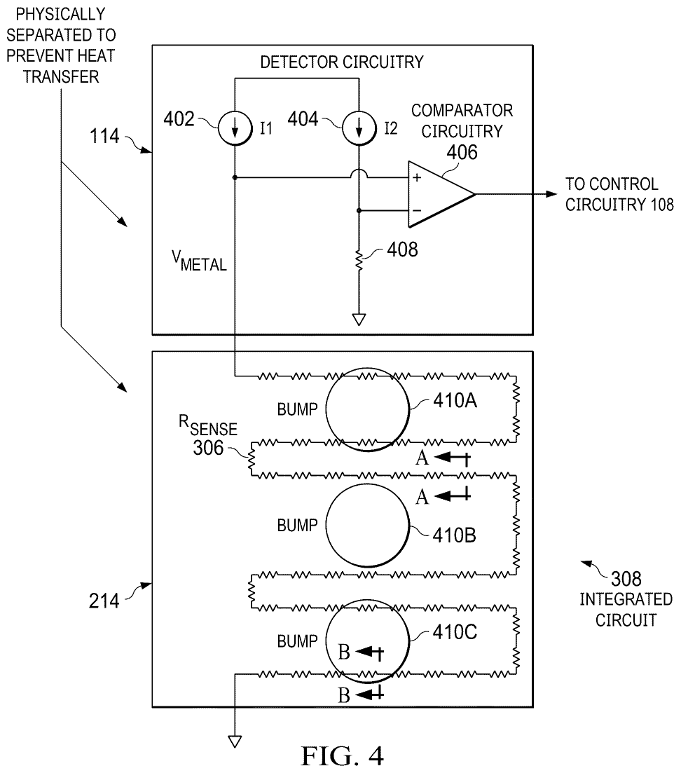

The detector circuitry 114 has a first terminal coupled to the control circuitry 108. The detector circuitry 114 detects the temperature of one or more components within the TX circuitry 112 without being electrically coupled to the TX circuitry 112. The detector circuitry 114 is described further in connection with FIG. 4.

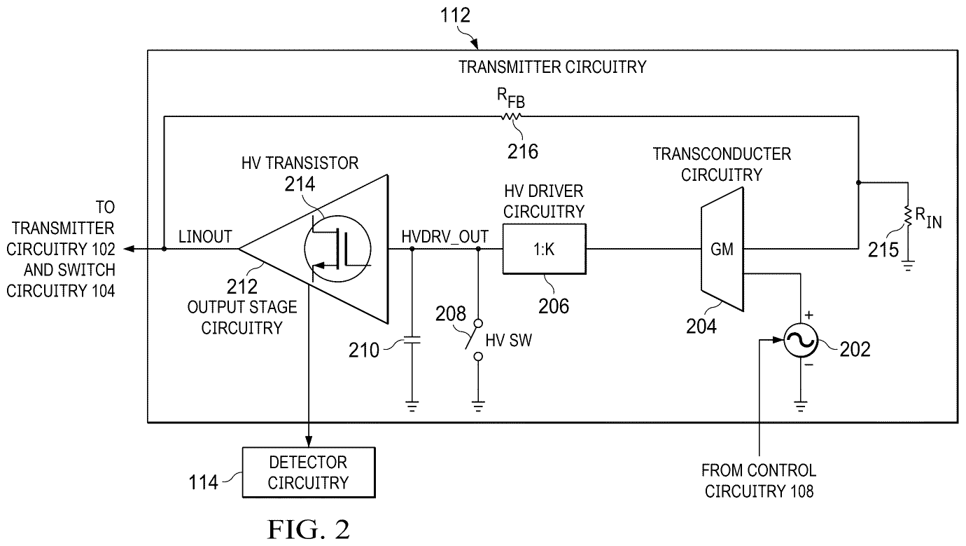

FIG. 2 is a block diagram of an example implementation of the TX circuitry 112 of FIG. 1. In the example of FIG. 2, the TX circuitry 112 includes a power source 202, transconducter circuitry 204, high voltage (HV) driver circuitry 206, a HV switch 208, a capacitor 210, output stage circuitry 212, an input resistor (R_{IN}) 215, a feedback resistor (R_{FB}) 216.

The power source 202 has a first terminal coupled to the transconducter circuitry 204, a second terminal coupled to ground, and a control terminal coupled to the control circuitry 108. The power source 202 provides a voltage to the transconducter circuitry 204 responsive to a signal from the control circuitry 108.

The transconducter circuitry 204 has a first terminal coupled to the power source 202. The transconducter circuitry 204 also has a second terminal that is coupled to both R_{IN} 215 and R_{FB} 216. The transconducter circuitry 204 presents low impedance at virtual ground and senses error current.

The HV driver circuitry 206 has a first terminal coupled to the transconducter circuitry 204 and a second terminal. The HV driver circuitry 206 increases the gain of the sensed transconducter error current by a factor of K. The voltage and current output by the HV driver circuitry 206, e.g., at the second terminal, is labelled in the example of FIG. 2 as the HVDRV_OUT node.

The HV switch 208 has a first terminal coupled to the HVDRV_OUT node and a second terminal coupled to ground. Similarly, the capacitor 210 has a first terminal coupled to the HVDRV_OUT node and a second terminal coupled to ground. The HV switch 208 and the capacitor 210 help reduce glitching that can occur when the TX circuitry 112 powers ON or OFF.

The output stage circuitry 212 is an electrical circuit having a first terminal that is coupled to the HVDRV_OUT node and a second terminal that is coupled to the switch circuitry 104 of FIG. 1. The voltage and current at the output, e.g., the second terminal, of the output stage circuitry 212 is labelled in the example of FIG. 2 as the LINOUT node. The output stage circuitry 212 acts as a voltage buffer that drives the transducer circuitry 102 as a load.

The output stage circuitry 212 supports voltage buffer operations by implementing a circuit architecture that is designed to provide a sourcing and sinking current of approximately +/−3 Amps (A). The circuit architecture includes the HV transistor 214, which experiences large peak-to-peak voltage (V_{PP}) swings and dissipates large amounts of power during such voltage buffer operations. For example, during elastography mode with a transducer load of 300 pico-Farads (pF), the HV transistor 214 may experience approximately 120 V_{PP} and dissipate approximately 10 Watts. Accordingly, the HV transistor 214 generates heat during some operations, e.g., elastography mode, which could damage the output stage circuitry 212 or, more generally, the TX circuitry 112.

In the examples described herein, the HV transistor 214 is a n-channel metal-oxide semiconductor field-effect transistor (MOSFET). Alternatively, the HV transistor 214 may be an n-channel field-effect transistor (FET), an n-channel insulated-gate bipolar transistor (IGBT), an n-channel junction field effect transistor (JFET), an NPN bipolar junction transistors (BJT) or, with slight modifications, p-type equivalent devices. The HV transistor 214 may be a depletion mode device, a drain-extended device, an enhancement mode device, a natural transistor, or another type of device structure transistor. Furthermore, the HV transistor 214 may be implemented in/over a silicon substrate (Si), a silicon carbide substrate (SiC), a gallium nitride substrate (GaN) or a gallium arsenide substrate (GaAs).

The detector circuitry 114 measures the temperature of the HV transistor 214 and reports the measurement to the control circuitry 108. The detector circuitry 114 is described further in connection with FIG. 4.

R_{IN} 215 has a first terminal coupled to the second terminal of the transconducter circuitry 204 and a second terminal coupled to ground. R_{FB} 216 has a first terminal coupled to the second terminal of the transconducter circuitry 204 and a second terminal coupled to the output of the output stage circuitry 212.

FIG. 3A is a top-down view of an Integrated Circuit (IC) that implements the High Voltage (HV) transistor and detector circuitry of FIG. 2 using a previous approach. FIG. 3A shows a top-down view of an IC layer 300. Accordingly, the components and materials shown in FIG. 3A are implemented at a uniform depth within the same layer of an IC.

As used above and herein, a layer of an IC refers to a three-dimensional region that varies in the x and y dimensions but is constant and uniform in the z dimension. Accordingly, multiple components and materials may be implemented within a single layer of an IC. Furthermore, components and materials within the same layer exist at the same depth, while components and materials from different layers are positioned over or beneath one another. When referencing at least one of a semiconductor device, e.g., a transistor, a semiconductor die containing a semiconductor device, or an integrated circuit (IC) package containing a semiconductor die during fabrication or manufacturing, “over”, “beneath”, and “below” are not with reference to Earth, but instead are with reference to an underlying substrate from which relevant components are fabricated, assembled, mounted, supported, or otherwise provided. Thus, as used herein and unless otherwise stated or implied from the context, a first component within a semiconductor die, e.g., a transistor or other semiconductor device, is “over” a second component within the semiconductor die when the first component is farther away from a substrate, e.g., a semiconductor wafer, during fabrication/manufacturing than the second component on which the two components are fabricated or otherwise provided. Semiconductor devices are often used in orientation different than their orientation during fabrication. A first part can be over or below a second part with one or more of: other parts therebetween, without other parts therebetween, with the first and second parts touching, or without the first and second parts being in direct contact with one another. As used in this patent, stating that any part, e.g., a layer, film, area, region, or plate, is in any way “on” another part, indicates that the referenced part is in direct contact with the other part and that no intermediate parts exist between the two parts.

The IC layer 300 shown in FIG. 3A includes a first region 302 and a second region 304. The first region 302 represents the area of a high voltage transistor within ultrasound transmitter circuitry. The region 302 has multiple fingers that increase the amount of current through the high voltage transistor by providing multiple parallel paths through which current can flow.

The second region 304 represents the area of detector circuitry implemented using previous approaches, e.g., with a bandgap core. While both the first region 302 and second region 304 are implemented in the same layer, they are separated by a buried oxide (BOX) material that is also within the same layer. In FIG. 3A, the BOX material is represented as gaps in the x and y dimensions between the first region 302 and the second region 304.

The BOX material implements the SOI architecture by galvanically separating the high voltage transistor of the first region 302 from other components within the IC. However, the BOX material also physically separates the first region 302 from the detector circuitry in the second region 304. Accordingly, the BOX material is a poor conductor of heat between the first region 302 and second region 304. Therefore, detector circuitry with components implemented in the same layer as the high voltage transistor are unable to accurately measure the high voltage transistor in SOI architectures. The BOX material is described further in connection with FIGS. 5 and 7.

FIG. 3B is a top-down view of an Integrated Circuit (IC) that implements the HV transistor 214 used in examples described herein. FIG. 3B shows a portion of an example IC 308. The IC 308 includes the HV transistor 214 of FIG. 2 and an example sense resistor 306 (labeled herein as R_{SENSE} 306).

Like the region 302 in in FIG. 3A, FIG. 3B shows that the HV transistor 214 has multiple fingers that parallelize and increase the flow of current. But unlike FIG. 3A, the IC layer that implements the HV transistor 214 does not include components or materials used to detect the temperature of the HV transistor 214. Instead, R_{SENSE} 306 is implemented over the HV transistor 214 in a different, second layer. As described further in connection with FIGS. 4-9, R_{SENSE} 306 is coupled to the detector circuitry 114 and is used to measure, e.g., to sense, the temperature of the HV transistor 214.

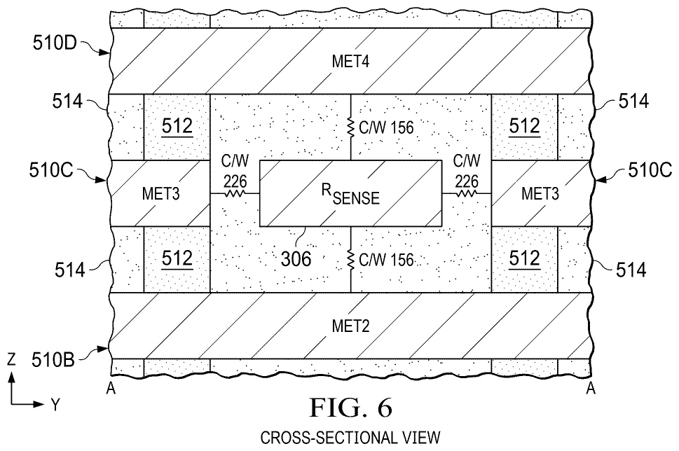

Notably, the BOX material does not prevent heat flow from the HV transistor 214 to R_{SENSE} 306 because the BOX material is not implemented over the HV transistor 214. Furthermore, the example FIG. 3B shows that R_{SENSE} 306 is implemented in a raster pattern over the region that implements the HV transistor 214. In other examples, a R_{SENSE} 306 is implemented in a different pattern over the region that implements the HV transistor 214. Thus, an average temperature reading obtained using R_{SENSE} 306 is responsive to localized temperature variations that occur in different portions of the HV transistor 214. More generally, positioning R_{SENSE} 306 over a heat generating component in an SOI architecture, e.g., the HV transistor 214, enables more accurate temperature measurement than previous approaches that include measurement components, e.g., the bandgap core, in the same layer as the heat generating component. The transfer of heat into R_{SENSE} 306 and positioning of the BOX material are described further in connection with FIGS. 5 and 6.

FIG. 4 is a schematic diagram of an example implementation of the detector circuitry 114 and IC 308 of FIGS. 1 and 3B. The IC 308 includes the HV transistor 214, R_{SENSE} 306, and example bumps 410A, 410B, 410C (collectively referred to as bumps 410). The detector circuitry 114 includes example current sources 402 and 404, example comparator circuitry 406, and an example resistor 408 (referred to herein as R₄₀₈).

FIG. 4 shows that the HV transistor 214 has a first terminal coupled to ground and a second terminal coupled to the comparator circuitry 406. In the example of the FIG. 4, the current source 402 pushes a first amount of current, I₁, through R_{SENSE} 306. Accordingly, the voltage at the positive terminal of the comparator circuitry 406, labeled V_{METAL}, is given by equation (1):

$${V}_{METAL} = {I}_{1} ( {R}_{SENSE 306} ) \qquad ( 1 )$$

Similarly, the voltage of the negative terminal of the comparator circuitry, referred to

herein as V_{THRESHOLD}, is given by equation (2):

$${V}_{THRESHOLD} = {I}_{2} ( {R}_{408} ) \qquad ( 2 )$$

Notably, R_{SENSE} 306 is implemented with a material whose resistance value changes proportionally with its temperature. Thus, when the temperature of the HV transistor 214 increases, the temperature of R_{SENSE} 306 increases, the resistance value of R_{SENSE} 306 increases, and the value of V_{METAL} increases. Furthermore, while the detector circuitry 114 and the IC 308 are shown adjacent to one another in FIG. 4, the two devices are physically separated from one another in practice. Thus, an increase in the temperature of the HV transistor 214 affects the resistance value of R_{SENSE} 306 but does not affect the resistance value of R₄₀₈.

The comparator circuitry 406 is triggered, e.g., changes an output voltage from a logical ‘1’ to a logical ‘0’ or vice versa, when V_{METAL}≥V_{THRESHOLD}. In the example of FIG. 4, the values of I₁ and I₂ are approximately equal. By substituting equations (1) and (2) into the foregoing inequality and cancelling out the current terms, the expression R_{SENSE} 306≥R₄₀₈ also describes when the comparator circuitry 406 triggers. Accordingly, a trigger by the comparator circuitry 406 indicates the temperature of the HV transistor 214 has exceeded a threshold temperature and is therefore overheating. A designer or manufacturer of the detector circuitry 114 can select the threshold temperature by adjusting the value of the resistor 408, thereby adjusting V_{THRESHOLD}. Accordingly, the resistor 408 can be adjusted so that crossing the overheating threshold indicates the components are currently suffering damage or behaving unexpectedly, the components are at risk of suffering damage or behaving unexpectedly, etc.

The comparator circuitry 406 has an output that is coupled to the control circuitry 108. Accordingly, the control circuitry 108 is notified when the comparator circuitry 406 and can take actions to stop or mitigate the overheating. Such actions include but are not limited to instructing the TX circuitry 112 to reduce the number of transmissions in elastography mode.

In the examples described herein, a Printed Circuit (PCB) implements one or more of the components shown in FIG. 2. Such components may include but are not limited to other components of the output stage circuitry 212 or, more generally, other components of the TX circuitry 112. The HV transistor 214 can transmit signals to, and receive signals from, the foregoing components because the IC 308 is coupled to the PCB in a Flip Chip-Ball Grid Array (FC-BGA) configuration. The bumps 410 refer to spheres of semiconductor material, e.g., silicon, which are used in the FC-BGA configuration to physically and electrically couple the rest of the IC 308 to the PCB. While FIG. 4 shows three bumps 410A, 410B, 410C for simplicity, the IC 308 may include any number of bumps 410 in practice.

The bumps 410 are implemented over both the layer that includes the HV transistor 214 and the layer that includes R_{SENSE} 306. In some examples, approximately 16% of the area of the HV transistor 214 is directly beneath one of the bumps 410. In the example of FIG. 4, portions of R_{SENSE} 306 are directly beneath the bumps 410A and 410C but are not directly beneath the bump 410B. A cross-sectional view of the perspective labeled ‘A’ in FIGS. 3B and 4, in which there is no bump implemented directly above R_{SENSE} 306, is shown in FIGS. 5 and 6. A cross-sectional view of the perspective labeled ‘B’ in FIGS. 3B and 4, in which the bump 410C is implemented directly above R_{SENSE} 306, is shown in FIG. 7.

FIG. 5 is a cross-sectional view of the IC 308 of FIG. 3B. In the example of FIG. 5, the IC 308 includes a mold compound 502, a substrate 504, a buried oxide (BOX) region 506, the HV transistor 214, R_{SENSE} 306, five metals (referred to herein as MET1, MET2, MET3,MET4, and MET5), vias 512, an oxide material 514, a mold compound 516, and a package stack 518. The example of FIG. 5 also includes a signal layer 508, metal layers 510A, 510B, 510C, 510D, 510E (collectively referred to as metal layers 510), and a region 520.

The mold compounds 502 and 516 refer to plastics that are used to encapsulate electronic components, e.g., the HV transistor 214 in FIG. 5. In keeping with the usage of the terms “over” and “below” throughout the examples described herein, the mold compound 502 is considered at or near the bottom of the IC 308 while the mold compound 516 is considered at or near the top of the IC 308.

FIG. 5 shows the substrate 504 is implemented on the mold compound 502. The substrate 504 acts as a structural foundation that other parts of the IC 308 are built over during fabrication. The substrate 504 may also serve as a ground plane for various components of the IC 308. In the example of FIG. 4, the substrate 504 is implemented with silicon. In other examples, the substrate 504 may be implemented using a different type of semiconductive material.

The HV transistor 214 is also implemented using a semiconductive material, e.g., silicon. The HV transistor 214 and the substrate 504 are doped such that, if the HV transistor 214 was implemented on the substrate 504, the HV transistor 214 would be electrically coupled to the ground plane. Such a connection would prevent the galvanic isolation between the HV transistor 214 and the other components of the IC 308. Instead, FIG. 5 shows the BOX region 506 is implemented on the substrate 504 and the HV transistor 214 is implemented on the BOX region 506. The material in the BOX region 506 is a strong insulator that prevents significant current flow and significant heat transfer. That is, the amount of heat that transfers from a first layer, through the BOX region, and to an adjacent layer is comparatively low to the amount of heat that uses a different medium to transfer from the first layer to the adjacent layer. Thus, HV transistor 214 is implemented over the substrate 504 but not electrically coupled to the substrate 504.

The HV transistor 214 is also implemented in the signal layer 508. Accordingly, the a temperature of the silicon signal layer changes responsive to operations performed by the HV transistor 214. In general, the signal layer 508 refers to the depth at which various transistors within the IC 308 may be implemented. Therefore, a cross-sectional view of an IC that implements bandgap core circuitry would show the measurement circuit in a location such as the region 520: that is, within the signal layer 508 and at the same depth as the HV transistor 214. However, in the example of FIG. 5, the BOX region 506 extends into the signal layer 508 in a manner that physically separates and galvanically isolates the HV transistor 214 from the region 520. Thus, the bandgap core is unable to accurately measure temperature in an IC that uses a BOX material despite being in the same layer as the heat generating component.

Within the metal layers 510, the regions of MET1-MET5 transport various electrical signals between the HV transistor 214 and other electrical components. Thus, some regions of MET1-MET5 may implement interconnects, wires, etc. Signals travel between such metal regions and the HV transistor 214 using the vias 512. FIG. 5 shows that within the metal layers 510, the space between the various metal regions are filled with the oxide material 514. In some examples, the metal layers 510 and the signal layer 508 are fabricated using a Back end of Line (BEOL) technique. While the example of FIG. 5 includes five metal layers 510A-510E, in general the IC 308 may implement any number of metal layers 510. Furthermore, the MET1-MET5 metals may refer to any type(s) of metal. Any subset or grouping of the MET1-MET5 metals may refer to the same type of metal alloy or refer to different types of metal alloy.

In the example of FIG. 5, R_{SENSE} 306 is a strip of MET3 cut within the metal layer 510C. In other examples, R_{SENSE} 306 is implemented in a different one of the metal layers 510. In general, the sensitivity of R_{SENSE} 306 decreases when the resistor is implemented closer to the mold compound 516 because a nonzero amount of heat is absorbed by the vias 512 as heat flows upwards and away from the signal layer 508. However, because the vias 512 are designed to provide strong electrical connections to the metal layers 510, the amount of heat absorbed by the vias 512 is generally considered negligible. Thus, in some examples such as FIG. 5, a designer or manufacturer of the IC 308 may implement R_{SENSE} 306 in one of the intermediate metal layers 510, and still achieve highly accurate temperature measurements, because the complexity of the signal routing prevents R_{SENSE} 306 from being implemented in a metal layer that is closer to the HV transistor 214. The heat flow through the vias 512, the MET1-MET5 regions, and the oxide material 514 are described further in connection with FIG. 6.

R_{SENSE} 306 is not in direct physical contact with the vias 512 or any of the other regions of MET1-MET5. Furthermore, R_{SENSE} 306 is not electrically coupled to the HV transistor 214 or any of the output stage circuitry 212. Accordingly, the metal layer 510C includes a first metal region, e.g., an interconnect or wire, which is electrically coupled to the signal layer 508 and a second metal region, e.g., R_{SENSE} 306, that is electrically decoupled from the signal layer 508. Thus, R_{SENSE} 306 absorbs heat from the surrounding metals and measures the temperature of a heat-generating component independently of the operation of the operation of the heat-generating component. In some examples, the first metal region and the second metal region of the metal layer 510C are implemented with the same type of metal. In other examples, the first metal region and the second metal region of the metal layer 510C are implemented with different type of metals.

The mold compound 516 is implemented on the metal layers 510, and the package stack 518 is implemented on the mold compound 516. The package stack 518 represents a substrate material that enables components from the IC 308 to couple to an external PCB.

FIG. 6 is an example representation of heat transfer between the HV transistor, metal layers, and sense resistor of FIG. 5. FIG. 6 includes MET2, MET3, and MET4 regions, the metal layers 510B, 510C, and 510D, R_{SENSE} 306, the vias 512, and the oxide material 514. FIG. 6 represents a subset of the cross-sectional view of FIG. 5.

As described with reference to FIG. 5, the heat absorbed through the vias 512 can be considered negligible because the vias 512 are strong electrical conductors designed to transport a signal. Similarly, the heat absorbed through the MET1-MET5 regions can also be considered negligible because they are also strong electrical conductors designed to transport a signal. Thus, the temperature of the metal strips and the vias 512 that surround R_{SENSE} 306 have a temperature that is approximately the same temperature as the HV transistor 214. Furthermore, R_{SENSE} 306 is surrounded by such metal in all three dimensions: FIG. 6 shows MET2 and MET4 regions surround R_{SENSE} 306 in the z dimension, FIG. 6 shows MET3 regions in the y dimension, and other regions of MET3 surround R_{SENSE} 306 in the x dimension. The other regions of MET3 are implemented in the metal layer 510C, either in-front of or behind the cross-sectional view of FIG. 6.

While R_{SENSE} 306 is surrounded by metal at the approximately the same temperature as the heat generating component, the heat must still flow through the oxide material 514. In FIG. 6, the thermal resistance of the oxide material 514 is modeled as resistors, where voltage at given node represents temperature and current flow represents the flow of heat. Accordingly, the thermal resistance between R_{SENSE} 306 and either the MET2 or MET4 regions is approximately 156 degrees Celsius per Watt (° C./W). Similarly, the thermal resistance between R_{SENSE} 306 and the MET3 regions is approximately 226° C. per Watt. Furthermore, because heat flows through the resistors in parallel, the equivalent thermal resistance between R_{SENSE} 306 and all adjacent metals is approximately 46° C./W (presuming the adjacent materials are at the same temperature). Additionally, R_{SENSE} 306 is not a significant source of heat storage because its thermal capacitance has a comparatively low value of approximately 1 nano-Joule per degree Celsius (nJ/° C.). Accordingly, the thermal time constant of R_{SENSE} 306 is lower, and R_{SENSE} 306 can track the temperature changes of the HV transistor 214 more accurately, than previous approaches.

The annotations of FIG. 6 show that, when compared to the interconnects and vias around it, the oxide material 514 is a poor thermal conductor. However, the oxide material 514 is a better thermal conductor than the BOX regions used in SOI architectures. For example, application of the same modeling described above to the previous approach of FIG. 3A shows that the thermal resistance between the heat generating component of region 302 and the detector circuitry of region 304 is approximately 485° C./W (compared to 46° C./W in the example of FIG. 6). Furthermore, the thermal capacitance of silicon (which is used to implement the detector circuitry) has a comparatively large value of 40 microjoules per degree Celsius (uJ/° C.). Thus, the BOX region 506 exhibits less heat transfer than the oxide material 514.

Heat flow into the detector circuitry of FIG. 3A also suffers because the heat can only arrive from a comparatively small number of places. The direction of heat is limited because the BOX material is in contact with the detector circuitry at several points below and to the sides of the circuit, effectively stopping heat flow in those directions. The limited number of directions for heat flow, high thermal capacitance of silicon, and high thermal resistance of the BOX material collectively lead to a high thermal time constant that prevent accurate temperature tracking. In contrast, R_{SENSE} 306 is implemented in a different layer than the HV transistor 214, thereby avoiding the BOX region 506 entirely and allowing heat to flow into the resistor from all directions. Such a configuration enables the temperature of R_{SENSE} 306 to match the average temperature of the surrounding MET regions. The additional directions for heat flow, low thermal capacitance of R_{SENSE} 306, and low thermal resistance of the oxide material 514 collectively enable examples described herein to measure the temperature of a heat generating component in a SOI architecture more accurately than previous approaches that implement detection components in the same layer as the heat generating component.

FIG. 7 is a cross-sectional view of the IC 308 labeled ‘B’ in FIG. 3B and FIG. 4. FIG. 7 where a silicon bump is implemented over the sense resistor. FIG. 7 includes the mold compound 502, the substrate 504, the BOX region 506, the signal layer 508, the metal layers 510, the HV transistor 214, R_{SENSE} 306, the MET1-MET5 regions, the vias 512, the oxide material 514, the mold compound 516, and the package stack 518, which have the same physical position and function as described in connection with FIG. 5. For example, both of FIG. 5 and FIG. 7 show the signal layer 508 includes the HV transistor 214 and a portion of the BOX region 506. FIG. 7 also includes the bump 410C.

R_{SENSE} 306 is able to track the temperature of the HV transistor 214 in part because the BOX region 506 and the mold compound 516 act as thermal insulators. The insulation leaves the materials in the metal layers 510, including R_{SENSE} 306, as one of a few places where heat from the HV transistor 214 can flow. However, in regions where one of the bumps 410 is implemented directly over R_{SENSE} 306, some signals travel from the HV transistor 214, through the metal layers 510 and bump 410C, and to the package stack 518. Such signals carry both current and heat. Thus, some heat that would have flowed into R_{SENSE} 306 in FIG. 5 flows instead into bump 410C in FIG. 7.

While the decreased flow of heat in FIG. 7 does influence the measurement of the detector circuitry 114, any loss of performance due to the bumps 410 is generally considered negligible. Thus, some manufacturers or designers may implement R_{SENSE} 306 directly under one or more of the bumps 410 due to the complexity of signal routing within the IC 308. Additionally, some manufacturers or designers may implement R_{SENSE} 306 in a manner that avoids the resistor being under one or more of the bumps 410 to increase the accuracy of measurement readings.

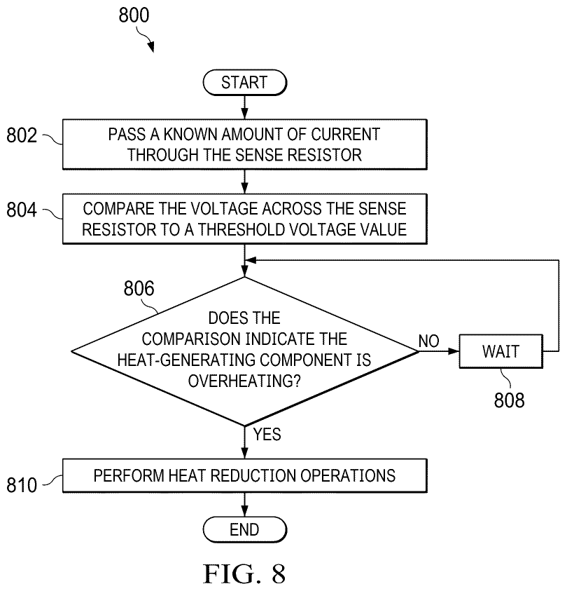

FIG. 8 is a flowchart representative of example machine-readable instructions and/or example operations 800 that may be at least one of executed, instantiated, or performed by the detector circuitry 114 and the control circuitry 108 of FIGS. 1 and 4. The example machine-readable instructions or the example operations 800 of FIG. 8 begin when the detector circuitry 114 passes a known amount of current through R_{SENSE} 306. (Block 802). The current of block 802 generates a sense voltage across R_{SENSE} 306. In FIG. 4, the current of block 802 is provided by current source 402 and the voltage is labeled V_{METAL}.

The detector circuitry 114 compares the voltage across R_{SENSE} 306 to a threshold voltage value. (Block 804). In the example of FIG. 4, the detector circuitry 114 generates the threshold voltage by passing an amount of current equal to that of block 802 through the resistor 408.

The control circuitry 108 determines whether the comparison of block 804 indicates the heat-generating component is overheating. (Block 806). In the foregoing examples, the heat-generating component is the HV transistor 214. In other examples, R_{SENSE} 306 is used to detect the temperature of a different component, including but not limited to components implemented outside the ultrasound system 100. The control circuitry 108 may determine the comparison of block 804 through any suitable technique, including but not limited to directly measuring the output of the comparator circuitry 406 of FIG. 4, receiving an interrupt or similar type of notification, etc.

If the comparison of block 804 indicates the heat generating component is at a safe temperature (Block 806: No), the control circuitry 108 waits an amount of time (Block 808) before control returns to block 806 and the result of a new comparison is evaluated. If instead the comparison of block 804 indicates the heat generating component is overheating (Block 806: Yes), the heat generating component mitigates performs heat reduction operations. (Block 810). The actions of block 810 refer to any that prevent, mitigate, or stop overheating. In the foregoing examples, the control circuitry 108 performs heat reduction operations by instructing the TX circuitry 112 to stop or reduce the amount of transmissions in elastography mode or stopping or reducing the amount of ultrasound transmissions in general.

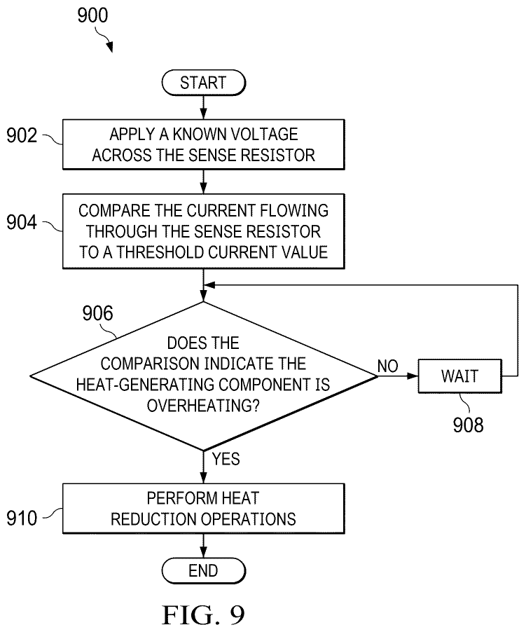

FIG. 9 is a flowchart representative of example machine-readable instructions or example operations that may be at least one of executed, instantiated, or performed the using the detector circuitry and the control circuitry of FIG. 1. The circuit diagram shown in FIG. 4 is just one example of how R_{SENSE} 306 may be used to measure the temperature of a heat-generating component. In other examples, the machine-readable instructions or the operations 900 begin when detector circuitry applies a known voltage across R_{SENSE} 306. (Block 902). The application of the known voltage generates an amount of current that is proportional to the temperature of the heat generating component. The detector circuitry then compares the current flowing through R_{SENSE} 306 to a threshold current value. (Block 904). In such examples, the control circuitry 108 executes blocks 906, 908, and 910 with respect to the comparison of block 904 in the same manner and technique that blocks 806, 808, and 810 are executed with respect to the comparison of block 804.

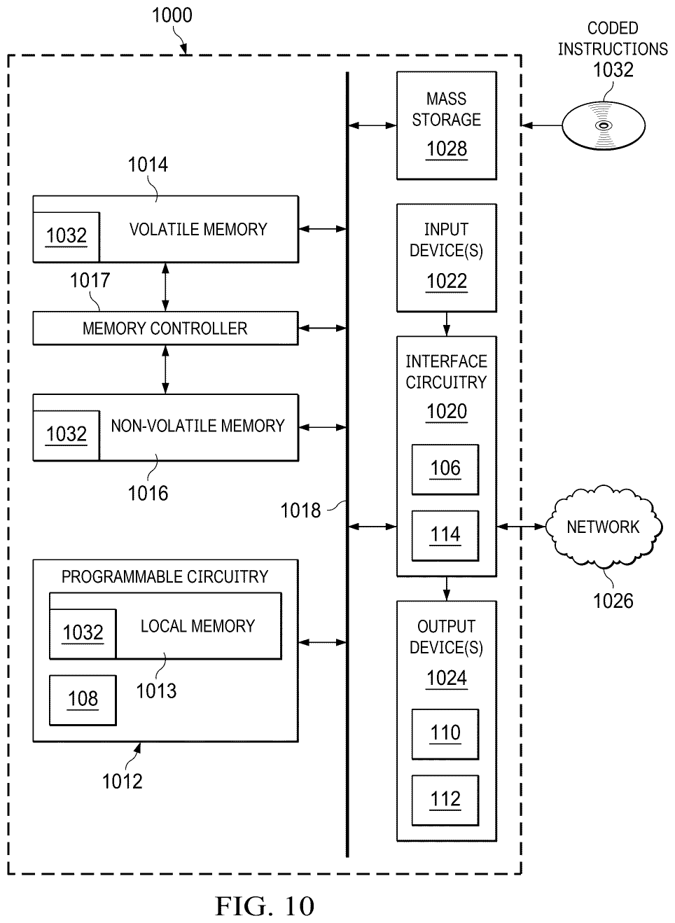

FIG. 10 is a block diagram of an example programmable circuitry platform 1000 structured to one or a combination of execute or instantiate one or more of the example machine-readable instructions or the example operations of FIGS. 8 and 9 to implement the control circuitry 108 of FIG. 1. The programmable circuitry platform 1000 can be, for example, a server, a personal computer, a workstation, a self-learning machine, e.g., a neural network, a mobile device, e.g., a cell phone, a smart phone, a tablet such as an iPad™, an ultrasound machine, an Internet appliance, or any other type of computing or electronic device.

The programmable circuitry platform 1000 of the illustrated example includes programmable circuitry 1012. The programmable circuitry 1012 of the illustrated example is hardware. For example, the programmable circuitry 1012 can be implemented by one or more integrated circuits, logic circuits, FPGAs, microprocessors, CPUs, GPUs, DSPs, or microcontrollers from any desired family or manufacturer. The programmable circuitry 1012 may be implemented by one or more semiconductor based, e.g., silicon based) devices. In this example, the programmable circuitry 1012 implements the control circuitry 108.

The programmable circuitry 1012 of the illustrated example includes a local memory 1013, e.g., a cache, registers, etc. The programmable circuitry 1012 of the illustrated example is in communication with main memory 1014, 1016, which includes a volatile memory 1014 and a non-volatile memory 1016, by a bus 1018. The volatile memory 1014 may be implemented by one or more Synchronous Dynamic Random Access Memory (SDRAM), Dynamic Random Access Memory (DRAM), RAMBUS® Dynamic Random Access Memory (RDRAM®), or any other type of RAM device. The non-volatile memory 1016 may be implemented by one or a combination of flash memory or any other desired type of memory device. Access to the main memory 1014, 1016 of the illustrated example is controlled by a memory controller 1017. In some examples, the memory controller 1017 may be implemented by one or more integrated circuits, logic circuits, microcontrollers from any desired family or manufacturer, or any other type of circuitry to manage the flow of data going to and from the main memory 1014, 1016.

The programmable circuitry platform 1000 of the illustrated example also includes interface circuitry 1020. The interface circuitry 1020 may be implemented by hardware in according to any type of interface standard, such as an Ethernet interface, a universal serial bus (USB) interface, a Bluetooth® interface, a near field communication (NFC) interface, a Peripheral Component Interconnect (PCI) interface, or a Peripheral Component Interconnect Express (PCIe) interface.

In the illustrated example, one or more input devices 1022 are connected to the interface circuitry 1020. The input device(s) 1022 permit(s) a user, e.g., a human user, a machine user, etc.) to enter one of or a combination of data or commands into the programmable circuitry 1012. The input device(s) 1022 can be implemented by, for example, the RX AFE circuitry 106, the detector circuitry 114, one of or a combination of an audio sensor, a microphone, a camera (still or video), a keyboard, a button, a mouse, a touchscreen, a trackpad, a trackball, an isopoint device, or a voice recognition system.

One or more output devices 1024 are also connected to the interface circuitry 1020 of the illustrated example. The output device(s) 1024 can be implemented, for example, by one of or a combination of the display 110, (e.g., a light emitting diode (LED), an organic light emitting diode (OLED), a liquid crystal display (LCD), a cathode ray tube (CRT) display, an in-place switching (IPS) display, a touchscreen, etc.), the TX circuitry 112, a tactile output device, a printer, or speaker. The interface circuitry 1020 of the illustrated example, thus, includes one of or a combination of a graphics driver card, a graphics driver chip, or graphics processor circuitry such as a GPU.

The interface circuitry 1020 of the illustrated example also includes a communication device such as one of or a combination of a transmitter, a receiver, a transceiver, a modem, a residential gateway, a wireless access point, or a network interface to facilitate exchange of data with external machines (e.g., computing devices of any kind) by a network 1026. The communication can be by, for example, an Ethernet connection, a digital subscriber line (DSL) connection, a telephone line connection, a coaxial cable system, a satellite system, a beyond-line-of-sight wireless system, a line-of-sight wireless system, a cellular telephone system, an optical connection, etc.

The programmable circuitry platform 1000 of the illustrated example also includes one or more mass storage discs or devices 1028 to store one or more of firmware, software, or data. Examples of such mass storage discs or devices 1028 include one or more magnetic storage devices (e.g., floppy disk, drives, HDDs, etc.), optical storage devices (e.g., Blu-ray disks, CDs, DVDs, etc.), RAID systems, or solid-state storage discs or devices such as flash memory devices and SSDs.

The machine-readable instructions 1032, which may be implemented by the machine-readable instructions of FIGS. 8 and 9, may be stored in one of or a combination of the mass storage device 1028, in the volatile memory 1014, in the non-volatile memory 1016, or in at least one non-transitory computer readable storage medium such as a CD or DVD which may be removable.

While an example manner of implementing the control circuitry 108 of FIG. 1 is illustrated in FIG. 1, one or more of the elements, processes, or devices illustrated in FIG. 1 may be combined, divided, re-arranged, omitted, eliminated, or implemented in any other way. Further, the control circuitry 108 of FIG. 1 may be implemented by hardware alone or by hardware in combination with software and firmware. Thus, for example, the control circuitry 108 could be implemented by programmable circuitry in combination with one or more machine-readable instructions (e.g., firmware or software), processor circuitry, analog circuit(s), digital circuit(s), logic circuit(s), programmable processor(s), programmable microcontroller(s), graphics processing unit(s) (GPU(s)), digital signal processor(s) (DSP(s)), ASIC(s), programmable logic device(s) (PLD(s)), or field programmable logic device(s) (FPLD(s)) such as FPGAs. Further still, the example control circuitry 108 of FIG. 1 may include one or more elements, processes, or devices in addition to, or instead of, those illustrated in FIG. 1 or may include more than one of any or all of the illustrated elements, processes and devices.

Flowcharts representative of example machine-readable instructions, which may be executed by programmable circuitry to at least one of implement or instantiate the control circuitry 108 and detector circuitry 114 of FIG. 1 or representative of example operations which may be performed by programmable circuitry to at least one of implement or instantiate the control circuitry 108 and detector circuitry 114 of FIG. 1, are shown in FIGS. 8 and 9. The machine-readable instructions may be one or more executable programs or portion(s) of one or more executable programs for execution by programmable circuitry such as the programmable circuitry 1012 shown in the example programmable circuitry platform 1000 described in connection with FIG. 10 and may be one or more function(s) or portion(s) of functions to be performed by the example programmable circuitry (e.g., an FPGA). In some examples, the machine-readable instructions control an operation, a task, etc., to be carried out or performed in an automated manner in the real-world. As used herein, “automated” means without human involvement.

The program may be embodied in instructions (e.g., software and/or firmware) stored in one or more non-transitory computer readable and/or machine-readable storage medium such as one of or a combination of cache memory, a magnetic-storage device or disk (e.g., a floppy disk, a Hard Disk Drive (HDD), etc.), an optical-storage device or disk (e.g., a Blu-ray disk, a Compact Disk (CD), a Digital Versatile Disk (DVD), etc.), a Redundant Array of Independent Disks (RAID), a register, ROM, a solid-state drive (SSD), SSD memory, non-volatile memory (e.g., electrically erasable programmable read-only memory (EEPROM), flash memory, etc.), volatile memory (e.g., Random Access Memory (RAM) of any type, etc.), or any other storage device or storage disk. The instructions of the non-transitory computer readable and/or machine-readable medium may program or be executed by programmable circuitry located in one or more hardware devices, but the entire program or parts thereof could alternatively be executed or instantiated by one or more hardware devices other than the programmable circuitry or embodied in dedicated hardware. The machine-readable instructions may be distributed across multiple hardware devices or executed by two or more hardware devices (e.g., a server and a client hardware device). For example, the client hardware device may be implemented by an endpoint client hardware device (e.g., a hardware device associated with a human and/or machine user) or an intermediate client hardware device gateway (e.g., a radio access network (RAN)) that may facilitate communication between a server and an endpoint client hardware device. Similarly, the non-transitory computer readable storage medium may include one or more mediums. Further, although the example program is described with reference to the flowchart(s) illustrated in FIGS. 8 and 9, many other methods of implementing the example control circuitry 108 may alternatively be used. For example, the order of execution of the blocks of the flowchart(s) may be changed, or some of the blocks described may be changed, eliminated, or combined. Also or alternatively, any or all of the blocks of the flow chart may be implemented by one or more hardware circuits (e.g., processor circuitry, discrete, integrated analog and/or digital circuitry, an FPGA, an ASIC, a comparator, an operational-amplifier (op-amp), a logic circuit, etc.) structured to perform the corresponding operation without executing software or firmware. The programmable circuitry may be distributed in different network locations or local to one or more hardware devices (e.g., a single-core processor (e.g., a single core CPU), a multi-core processor (e.g., a multi-core CPU, an XPU, etc.)). For example, the programmable circuitry may be one of or a combination of a CPU or an FPGA located in the same package (e.g., the same integrated circuit (IC) package or in two or more separate housings), one or more processors in a single machine, multiple processors distributed across multiple servers of a server rack, multiple processors distributed across one or more server racks, etc., or any combination(s) thereof.

The machine-readable instructions described herein may be stored in one or more of a compressed format, an encrypted format, a fragmented format, a compiled format, an executable format, a packaged format, etc. Machine readable instructions as described herein may be stored as data (e.g., computer-readable data, machine-readable data, one or more bits (e.g., one or more computer-readable bits, one or more machine-readable bits, etc.), a bitstream (e.g., a computer-readable bitstream, a machine-readable bitstream, etc.), etc.) or a data structure (e.g., as portion(s) of instructions, code, representations of code, etc.) that may be utilized to create, manufacture, or produce machine executable instructions. For example, the machine-readable instructions may be fragmented and stored in one or more storage devices, disks or computing devices (e.g., servers) located at the same or different locations of a network or collection of networks (e.g., in the cloud, in edge devices, etc.). The machine-readable instructions may require one or more of installation, modification, adaptation, updating, combining, supplementing, configuring, decryption, decompression, unpacking, distribution, reassignment, compilation, etc., so they can be directly readable, interpretable, or executable by a computing device or other machine. For example, the machine-readable instructions may be stored in multiple parts, which are individually compressed, encrypted, or stored in separate computing devices, such that the parts when decrypted, decompressed, or combined form a set of one or more computer-executable or machine executable instructions that implement one or more functions or operations that may together form a program such as that described herein.

In another example, the machine-readable instructions may be stored in a state in which they may be read by programmable circuitry, but require addition of a library (e.g., a dynamic link library (DLL)), a software development kit (SDK), an application programming interface (API), etc., in order to execute the machine-readable instructions in a particular computing device or other device. In another example, the machine-readable instructions may need to be configured (e.g., settings stored, data input, network addresses recorded, etc.) before the machine-readable instructions or the corresponding program(s) can be executed in whole or in part. Thus, machine-readable, computer readable or machine-readable media, as used herein, may include one or a combination of instructions and program(s) regardless of the particular format or state of the machine-readable instructions or program(s).

The machine-readable instructions described herein can be represented by any past, present, or future instruction language, scripting language, programming language, etc. For example, the machine-readable instructions may be represented using any of the following languages: C, C++, Java, C #, Perl, Python, JavaScript, HyperText Markup Language (HTML), Structured Query Language (SQL), Swift, etc.

The example operations of FIGS. 8 and 9 may be implemented using executable instructions (e.g., computer readable and/or machine-readable instructions) stored in one or more non-transitory computer readable or machine-readable media. As used herein, the terms non-transitory computer readable medium, non-transitory computer readable storage medium, non-transitory machine-readable medium, and non-transitory machine-readable storage medium are expressly defined to include any type of computer readable storage device or storage disk and to exclude propagating signals and to exclude transmission media. Examples of such non-transitory computer readable medium, non-transitory computer readable storage medium, non-transitory machine-readable medium, or non-transitory machine-readable storage medium include one or more optical storage devices, magnetic storage devices, an HDD, a flash memory, a read-only memory (ROM), a CD, a DVD, a cache, a RAM of any type, a register, or any other storage device or storage disk in which information is stored for any duration (e.g., for extended time periods, permanently, for brief instances, for temporarily buffering, for caching of the information). As used herein, the terms “non-transitory computer readable storage device” and “non-transitory machine-readable storage device” are defined to include any physical (mechanical, magnetic, electromechanical, or electrical) hardware to retain information for a time period, but to exclude propagating signals and to exclude transmission media. Examples of non-transitory computer readable storage devices or non-transitory machine-readable storage devices include one or a combination of random-access memory of any type, read only memory of any type, solid state memory, flash memory, optical discs, magnetic disks, disk drives, or redundant array of independent disks (RAID) systems. As used herein, the term “device” refers to physical structure such as one of or a combination of mechanical, electromechanical, or electrical equipment, hardware, or circuitry that may or may not be configured by computer readable instructions, machine-readable instructions, etc., or manufactured to execute computer-readable instructions, machine-readable instructions, etc.

“Including” and “comprising” (and all forms and tenses thereof) are used herein to be open ended terms. Thus, whenever a claim employs any form of “include” or “comprise” (e.g., comprises, includes, comprising, including, having, etc.) as a preamble or within a claim recitation of any kind, additional elements, terms, etc., may be present without falling outside the scope of the corresponding claim or recitation. As used herein, when the phrase “at least” is used as the transition term in, for example, a preamble of a claim, it is open-ended in the same manner as the term “comprising” and “including” are open ended. The term “and/or” when used, for example, in a form such as A, B, and/or C refers to any combination or subset of A, B, C such as (1) A alone, (2) B alone, (3) C alone, (4) A with B, (5) A with C, (6) B with C, or (7) A with B and with C. As used herein in the context of describing structures, components, items, objects and things, the phrase “at least one of A and B” refers to implementations including any of (1) at least one A, (2) at least one B, or (3) at least one A and at least one B. Similarly, as used herein in the context of describing structures, components, items, objects and things, the phrase “at least one of A or B” refers to implementations including any of (1) at least one A, (2) at least one B, or (3) at least one A and at least one B. As used herein in the context of describing the performance or execution of processes, instructions, actions, activities, etc., the phrase “at least one of A and B” refers to implementations including any of (1) at least one A, (2) at least one B, or (3) at least one A and at least one B. Similarly, as used herein in the context of describing the performance or execution of processes, instructions, actions, activities, etc., the phrase “at least one of A or B” refers to implementations including any of (1) at least one A, (2) at least one B, or (3) at least one A and at least one B.

As used herein, singular references (e.g., “a,” “an,” “first,” “second,” etc.) do not exclude a plurality. The term “a” or “an” object, as used herein, refers to one or more of that object. The terms “a” (or “an”), “one or more,” and “at least one” are used interchangeably herein. Furthermore, although individually listed, a plurality of means, elements, or actions may be implemented by, e.g., the same entity or object. Also, although individual features may be included in different examples or claims, these may possibly be combined, and the inclusion in different examples or claims does not imply that a combination of features is at least one of not feasible or advantageous.

Unless specifically stated otherwise, descriptors such as “first,” “second,” “third,” etc., are used herein without imputing or otherwise indicating any meaning of priority, physical order, arrangement in a list, or ordering in any way, but are merely used as at least one of labels or arbitrary names to distinguish elements for ease of understanding the described examples. In some examples, the descriptor “first” may be used to refer to an element in the detailed description, while the same element may be referred to in a claim with a different descriptor such as “second” or “third.” In such instances, such descriptors are used merely for identifying those elements distinctly within the context of the discussion (e.g., within a claim) in which the elements might, for example, otherwise share a same name.

As used herein, “approximately” and “about” modify their subjects/values to recognize the potential presence of variations that occur in real world applications. For example, “approximately” and “about” may modify dimensions that may not be exact due to at least one of manufacturing tolerances or other real-world imperfections. For example, “approximately” and “about” may indicate such dimensions may be within a tolerance range of +/−10% unless otherwise specified herein.

As used herein, the phrase “in communication,” including variations thereof, encompasses one of or a combination of direct communication or indirect communication through one or more intermediary components, and does not require direct physical (e.g., wired) communication or constant communication, but rather also includes selective communication at least one of periodic intervals, scheduled intervals, aperiodic intervals, or one-time events.

As used herein integrated circuit/circuitry is defined as one or more semiconductor packages containing one or more circuit elements such as transistors, capacitors, inductors, resistors, current paths, diodes, etc. For example, an integrated circuit may be implemented as one or more of an ASIC, an FPGA, a chip, a microchip, programmable circuitry, a semiconductor substrate coupling multiple circuit elements, a system on chip (SoC), etc.

In this description, the term “couple” may cover connections, communications, or signal paths that enable a functional relationship consistent with this description. For example, if device A generates a signal to control device B to perform an action: (a) in a first example, device A is coupled to device B by direct connection; or (b) in a second example, device A is coupled to device B through intervening component C if intervening component C does not alter the functional relationship between device A and device B, such that device B is controlled by device A via the control signal generated by device A.

A device that is “configured to” perform a task or function may be configured (e.g., at least one of programmed or hardwired) at a time of manufacturing by a manufacturer to at least one of perform the function or be configurable (or re-configurable) by a user after manufacturing to perform the function/or other additional or alternative functions. The configuring may be through at least one of firmware or software programming of the device, through at least one of a construction or layout of hardware components and interconnections of the device, or a combination thereof.

As used herein, the terms “terminal,” “node,” “interconnection,” “pin” and “lead” are used interchangeably. Unless specifically stated to the contrary, these terms are generally used to mean an interconnection between or a terminus of a device element, a circuit element, an integrated circuit, a device or other electronics or semiconductor component.

In the description and claims, described “circuitry” may include one or more circuits. A circuit or device that is described herein as including certain components may instead be adapted to be coupled to those components to form the described circuitry or device. For example, a structure described as including one or more semiconductor elements (such as transistors), one or more passive elements (such as one of or a combination of resistors, capacitors, or inductors), or one or more sources (such as voltage and/or current sources) may instead include only the semiconductor elements within a single physical device (e.g., at least one of a semiconductor die or integrated circuit (IC) package) and may be adapted to be coupled to at least some of the passive elements or the sources to form the described structure either at a time of manufacture or after a time of manufacture, for example, by at least one of an end-user or a third-party.

Circuits described herein are reconfigurable to include the replaced components to provide functionality at least partially similar to functionality available prior to the component replacement. Components shown as resistors, unless otherwise stated, are generally representative of any one or more elements coupled in at least one of series or parallel to provide an amount of impedance represented by the shown resistor. For example, a resistor or capacitor shown and described herein as a single component may instead be multiple resistors or capacitors, respectively, coupled in parallel between the same nodes. For example, a resistor or capacitor shown and described herein as a single component may instead be multiple resistors or capacitors, respectively, coupled in series between the same two nodes as the single resistor or capacitor. While certain elements of the described examples are included in an integrated circuit and other elements are external to the integrated circuit, in other example embodiments, additional or fewer features may be incorporated into the integrated circuit. Furthermore, some or all of the features illustrated as being external to the integrated circuit may be included in the integrated circuit and some features illustrated as being internal to the integrated circuit may be incorporated outside of the integrated. As used herein, the term “integrated circuit” means one or more circuits that are at least one of: (i) incorporated in/over a semiconductor substrate; (ii) incorporated in a single semiconductor package; (iii) incorporated into the same module; or (iv) incorporated in/on the same printed circuit board.

Uses of the phrase “ground” in the foregoing description include at least one of a chassis ground, an Earth ground, a floating ground, a virtual ground, a digital ground, a common ground, or any other form of ground connection applicable to, or suitable for, the teachings of this description.

Modifications are possible in the described embodiments, and other embodiments are possible, within the scope of the claims.

From the foregoing, it will be appreciated that example systems, apparatus, articles of manufacture, and methods have been described that accurately measure the temperature of heat generating components within Silicon on Insulator architectures. Described systems, apparatus, articles of manufacture, and methods improve the efficiency of using a computing device by implementing a sense resistor in a different layer that is over the heat generating component, thereby avoiding the high thermal resistance of the buried oxide materials that exist beneath the heat generating component and in the same layer as the heat generating component. Described systems, apparatus, articles of manufacture, and methods are also directed to one or more improvement(s) in the operation of a machine such as a computer or other electronic, electromechanical, or mechanical device.

What is claimed is:

1. An integrated circuit comprising:
a silicon signal layer; and
a metal layer over the silicon signal layer, wherein the metal layer includes a first metal region that is electrically coupled to the silicon signal layer and a second metal region that is electrically decoupled from the silicon signal layer.

2. The integrated circuit of claim 1, wherein:
the silicon signal layer implements a component in an electrical circuit; and
a temperature of the silicon signal layer changes responsive to operations performed by the component.

3. The integrated circuit of claim 2, further including:
a substrate beneath the silicon signal layer; and
a buried oxide region implemented between the substrate and the silicon signal layer.

4. The integrated circuit of claim 3, wherein:
the component is a first component;
the first metal region is an interconnect or a wire; and
the first metal region couples the first component to a different component.

5. The integrated circuit of claim 2, wherein a resistance of the second metal region changes proportionally to the temperature of the silicon signal layer.

6. The integrated circuit of claim 1, wherein the first metal region and the second metal region are implemented using the same type of metal.

7. The integrated circuit of claim 1, wherein the metal layer is a first metal layer, and wherein the integrated circuit further includes:
a second metal layer implemented below the first metal layer and over the silicon signal layer; and
a third metal layer implemented over the first metal layer and the second metal layer.

8. The integrated circuit of claim 1, further including:
vias coupled to both the first metal region and the silicon signal layer; and
oxide material implemented: a) between the first metal region and the second metal region and b) between the second metal region and the silicon signal layer.

9. The integrated circuit of claim 8, further including a buried oxide region implemented beneath the silicon signal layer, the vias, and the oxide material.

10. The integrated circuit of claim 1, wherein:
the silicon signal layer includes a component that is implemented across an area of the integrated circuit at a uniform depth;
the component exhibits localized temperature variations across different portions of the area; and
the second metal region is implemented across the area in a pattern to measure a temperature of the component.

11. The integrated circuit of claim 10, further including a silicon bump implemented above the metal layer and within the area, the silicon bump coupled to the silicon signal layer through the first metal region.

12. The integrated circuit of claim 11, wherein the pattern is implemented so that the second metal region is not implemented directly below the silicon bump.

13. An integrated circuit comprising:
a silicon substrate;
a buried oxide region on the silicon substrate;
a silicon signal layer on the buried oxide region;
a metal layer over the silicon signal layer, wherein the metal layer includes:
a first metal region that is electrically coupled to the silicon signal layer; and
a second metal region that is electrically decoupled from the silicon signal layer; and
oxide material implemented: a) between the first metal region and second metal region and b) between the second metal region and the silicon signal layer.

14. The integrated circuit of claim 13, wherein the integrated circuit further includes:
a first mold compound below the silicon substrate;
a second mold compound over the metal layer; and
a package stack on the second mold compound.

15. An ultrasound device comprising:
an integrated circuit including:
a silicon substrate;
a buried oxide region on the silicon substrate;
a silicon signal layer on the buried oxide region, wherein the silicon signal layer includes a transistor;
a metal layer over the silicon signal layer, wherein the metal layer includes a first metal region that is electrically coupled to the transistor and a second metal region that is electrically decoupled from the transistor; and
oxide material implemented: a) between the first metal region and second metal region and b) between the second metal region and the silicon signal layer;
detector circuitry coupled to the second metal region, the detector circuitry configured to detect when a temperature of the transistor exceeds a threshold temperature; and
control circuitry coupled to the detector circuitry.

16. The ultrasound device of claim 15, wherein the detector circuitry is configured to:
pass a current through the second metal region to generate a sense voltage; and
compare the sense voltage to a threshold voltage.

17. The ultrasound device of claim 15, wherein the detector circuitry is configured to:
apply a voltage across the second metal region to generate a sense current; and
compare the sense current to a threshold current.

18. The ultrasound device of claim 15, further including:
a transducer configured to generate ultrasound waves; and
transmitter circuitry configured to excite the transducer, wherein the transistor is implemented within an output stage of the transmitter circuitry.

19. The ultrasound device of claim 18, wherein the control circuitry is configured to instruct the transmitter circuitry to excite the transducer in an elastography mode.

20. The ultrasound device of claim 19, wherein the control circuitry is configured to stop or reduce an amount of transmissions in the elastography mode responsive to the detection.
